(19) United States
(12) Patent Application Publication
(10) Pub. No.: US 2010/0081870 A1
(43) Pub. Date: Apr. 1, 2010

(54) APPARATUS FOR TRAINING THE MALE ABDOMINAL MUSCLES

(76) Inventor: Richard M. Lehmann, Bohl-Iggelheim (DE)

Correspondence Address: Frijouf Rust & Pyle, 201 East Davis Blvd, Tampa, FL, 33606, US

(21) Appl. No.: 12/312,750

(22) PCT Filed: Nov. 8, 2007
(86) PCT No.: PCT/DE2007/002013

§ 371 Date: May 26, 2009

(30) Foreign Application Priority Data
Nov. 30, 2006 (DE) 20 2006 018 166.0

(51) Int. Cl. A61F 5/00

(52) U.S. Cl. 600/38

(57) ABSTRACT

1. Apparatus for training the male abdominal musculature.

2.1 Apparatuses for training the male abdominal muscles are known which incorporate a vessel to accommodate the male member creating a seal. After generating negative pressure inside the vessel, a weight is attached to the same so that a training effect results from the required counteracting force. The object of the present invention is to improve the training effect of such known apparatuses.

2.2 According to the present invention, this problem is solved by designing the vessel to be short enough so that only the glans of the male member can be accommodated within it. This facilitates lateral training movements which are prevented by the rigidity of known longer vessels.

2.3 Muscle training

The invention concerns an apparatus to train the male abdominal muscles as well as stretching and expanding the male member, consisting of a pressure vessel to accommodate the male member creating a seal where negative pressure can be generated in the pressure vessel.

Such an apparatus is already known from U.S. Pat. No. 5,836,864 A. The apparatus described there mainly consists of a cylindrical vessel in a shape and size to suitable to accommodate a male member. A narrow section is provided at the insertion end so that a seal can be created by the inserted member, while a sliding element is provided at the end opposite this insertion point. The sliding element is equipped with a handle, to which a movable slider which also seals where it contacts the vessel is attached.

Once a male member is inserted into the vessel, the sealing effect at the insertion end allows negative pressure to be generated in the vessel by moving the sliding element away from the insertion end which, by generating negative pressure inside the male member which is not protected against the negative pressure in the vessel, results in higher blood pressure inside the male member. This results in an erection of the member, which constitutes the desired effect of the procedure.

In a further development of this concept, weights can be assigned to the supporting element so that training of the user's abdominal muscles can be achieved by lifting up the vessel—which is held in position on the member by negative pressure—along with the weights.

However, this additional training effect only applies to a limited set of musculature since sideways deviation of the member during training is made impossible by the vessel, which extends over the entire length of the member.

Causing the member to lengthen using a stretching apparatus, which encompasses the glans and is supported against the body thereby stretching the member lengthwise, is also a known procedure. While this type of apparatus does cause the member to lengthen, is simultaneously causes it to become thinner. The latter of the two preceding effects is undesirable.

Based on this background information, the object of the present invention is to create an apparatus to train the male abdominal muscles as well as stretching and expanding the male member, which expands the area of application of known training apparatuses and combines known apparatuses in a meaningful manner.

This is achieved with the help of an apparatus to train the male abdominal muscles as well as stretching and expanding the male member in accordance with the characteristics of the main claim. Meaningful further developments of the invention are derived from the subsidiary claims.

According to the present invention the apparatus in accordance with the characteristics of the main claim mainly consists of a pressure vessel to accommodate the male member, where the member is only accommodated in the pressure vessel up to and including the glans. The vessel is made of hard plastic, clear if applicable, in the form of a sealed, cylindrical hollow body. The seal is formed in part by the member inserted into the pressure vessel. Negative pressure is then generated inside the pressure vessel, which causes blood to flow into the spongy bodies of the member resulting in an erection. Because the member is only inserted into the vessel up to and including the glans, sideways movement of the member remains possible which facilitates training for additional sets of musculature. In order to conduct the aforementioned training, a supporting element is assigned to the pressure vessel. One or more training aids can be attached to this supporting element in order to exercise the abdominal muscles of the user.

The training exercise itself is conducted by, for example, completing a swinging motion with attached weights so that blood is massaged into the anterior section of the member and into the glans in particular. The effect of an increase in volume intended by nature is intensified by this additional flow of blood. With regular use, the ability of the areas subject to training to absorb blood is intensified so that thickening can be observed over time when the member is engorged with blood.

In order to generate negative pressure, a cap is assigned to the pressure vessel. Said cap is applied to the pressure vessel so that it is removable. The cap has an insertion opening through which the member can be inserted into the pressure vessel. Because this opening encompasses the member in a formfitting manner, the pressure vessel is sealed so that negative pressure can be generated.

It is advantageous if the cap is made of an elastic material, such as a foamed synthetic material, so that the member can be inserted but is also encompassed in a formfitting manner in this state which allows the pressure vessel to be sealed.

For a connection between the pressure vessel and the cap, the latter incorporates a connecting groove in the area of the exterior circumference of the cap into which the open end of the pressure vessel can be inserted. The material of the cap encompasses the edge of the pressure vessel, creating a seal, so that the degree of sealing required to generate negative pressure inside the pressure vessel is assured. During subsequent use, the cap and pressure vessel are held together by friction between the cap material in the area of the connecting groove and the pressure vessel as well as the negative pressure inside the pressure vessel.

In order to generate negative pressure inside the pressure vessel, a suction adapter to which a suction pump can be connected is attached to the pressure vessel—preferably in the area opposite to the cap. With the suction pump attached to the pressure vessel, the air inside the pressure vessel can be evacuated so that the desired negative pressure is generated. By turning off the suction pump or, if applicable, disconnecting the suction line or reversing the pump effect, the negative pressure can equalised and the applied apparatus can be removed by the user.

The supporting element to attach training aids is also preferably arranged on the end of the pressure vessel opposite the cap so that the attached training aids unfold their training effect with the greatest possible leverage. This effect is also supported by the short design and attachment only to the glans according to the present invention. In this manner, a greater training effect can be achieved with a low weight. The supporting element is preferably implemented as a supporting ring or supporting hook.

Favoured training aids include weights and expanders that can exert force on the musculature to be exercised. When attaching weights, this may be achieved using a supporting cable so that the weight can also generate additional sideways forces with oscillating movements in addition to the force of its weight. When using expanders, the energy is absorbed by the tension springs and, with the advantageous design of the pressure vessel according to the present invention, can be applied in various directions and especially also upward in contrast to the use of weights.

In concrete implementations the aforementioned training aids can also be attached to a supporting element underneath, which the user can exercise in a prone position. Such a supporting element may consist of a swivel arm to which an expander is mounted or to which one or more weights are attached with the help of pulleys. This also allows the training aids and in particular the weights to act against the effects of gravity.

The invention described above is explained in greater detail below using an implementation example shown in the drawing.

The figures show the following:

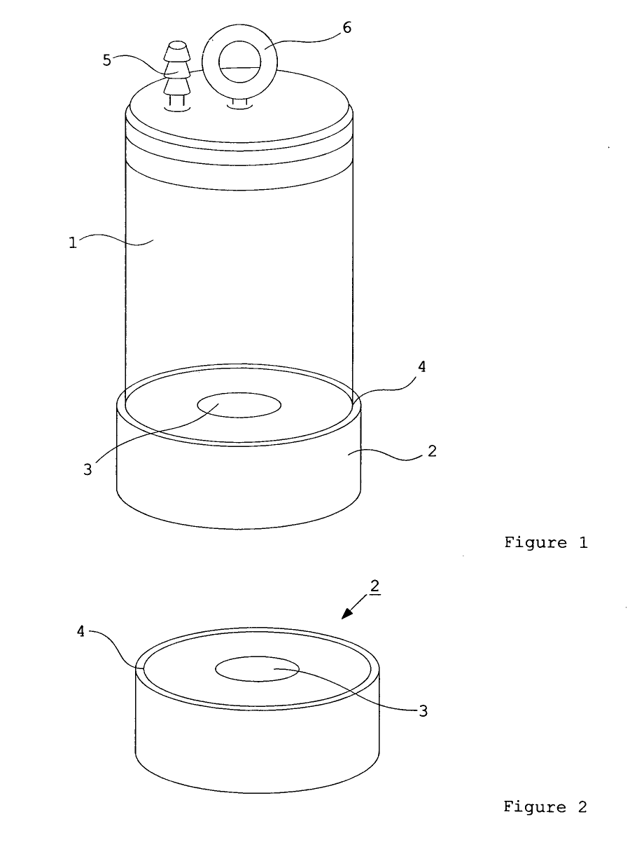

FIG. 1: A perspective drawing of an apparatus for training the abdominal musculature.

FIG. 2: A cap for the apparatus according to FIG. 1.

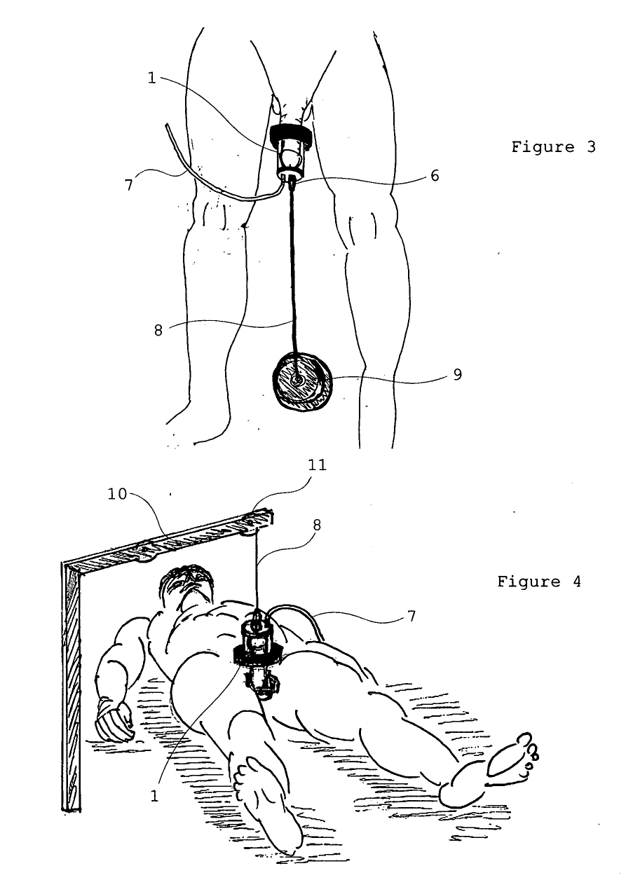

FIG. 3: A perspective drawing of the apparatus according to FIG. 1 in an application with a weight.

FIG. 4: The apparatus according to FIG. 1 in an application with a swivel arm serving as a supporting element.

FIG. 1 shows a pressure vessel to accommodate a male member up to and including the glans. The pressure vessel 1 is equipped with a suction pump to generate negative pressure, which can be connected to the pressure vessel 1 via a suction adapter 5. By evacuating the pressure vessel 1 after inserting a male member, blood pressure in the male member is increased causing an erection. The pressure vessel 1 is implemented in the form of a cylinder which is open on one end. The open end is closed by a cap 2. The pressure vessel also has a supporting element 6 to which at least one training aid can be attached. A training effect can be applied to the affected abdominal muscles with the help of the training aid(s). Because of the short design of the pressure vessel 1 and the fact that inserting the member up to and including the glans is sufficient, a training effect in the lateral direction is also facilitated.

FIG. 2 shows the cap 2 of the apparatus in FIG. 1, which is used to close off and seal the pressure vessel 1. By attaching the open end of the pressure vessel 1 to the cap 2 by means of a connecting groove 4, an encompassing connection is created which is initially held in place by friction and, after generating negative pressure, also by said negative pressure thereby creating a seal. Located in the middle of the cap 2 is an insertion opening 3 which allows the member of the user to be inserted into the pressure vessel 1. Insertion may be performed before or also after attaching the cap 2 to the pressure vessel 1.

FIG. 3 shows the apparatus in its intended state, where the pressure vessel 1 is attached to the cap 2 and the member of the user has been inserted into the interior of the pressure vessel 1 through the insertion opening 3 up to an including the glans. Subsequently a suction hose 7 was connected to the suction adapter 5 of the pressure vessel 1, connecting a suction pump to the pressure vessel 1. Negative pressure is generated inside the pressure vessel 1 by the suction action of the pump, leading to an erection of the member. In this state a training effect can be achieved by attaching a supporting cable 8 to the supporting element 6 on the pressure vessel 1 and a weight 9 to the supporting cable 8 so that traction is applied to the abdominal musculature of the user. The direction of the traction force and therefore the direction of the training effect can be varied by causing the weight 9 to oscillate sideways. Exercising additional musculature is facilitated by the short design which does not impede the lateral movement of the member. Evenly swinging the weight 9 back and forth massages blood into the glans leading to the enlargement of vessels. This additional engorgement with blood leads to the desired thickening of the member over time with simultaneous lengthening caused by the training aids applying traction force away from the body. It is also possible to combine the apparatus according to the present invention with known apparatuses for training purposes, that is, using them in succession in order to benefit equally from the different effects of the individual apparatuses. In this regard the different apparatuses are also combined with each other in sets so that the user can perform training in a targeted manner.

Another training option involves the user lying underneath a supporting element such as a swivel arm 10, where a supporting cable 8 is attached to the supporting element 6 of the attached pressure vessel 1. This supporting cable is guided over at least one pulley 10 arranged on the swivel arm so that, because of the reversing action, a weight attached to the supporting cable can pull in the direction of the force of gravity but cannot act upwards against this force.

Therefore an apparatus for training the male abdominal muscles as well as stretching and expanding the male member is described above, which extends and combines the area of application of known training apparatuses by making it possible to exercise the muscles in question by facilitating lateral freedom of movement.

### LIST OF REFERENCE SIGNS

1 Pressure vessel
2 Cap
3 Insertion opening
4 Connecting groove
5 Suction adapter
6 Supporting element
7 Suction hose
8 Supporting cable
9 Weight
10 Swivel arm
11 Pulley

What is claimed is:

1. Apparatus for training the male abdominal musculature, consisting of a pressure vessel (1) to accommodate a male member creating a seal, allowing negative pressure to be generated in the pressure vessel (1), wherein the member can only be inserted into the pressure vessel (1) up to and including the glans and the pressure vessel (1) has at least one supporting element (6).

2. (canceled)

3. (canceled)

4. (canceled)

5. (canceled)

6. (canceled)

7. (canceled)

8. (canceled)

9. (canceled)

10. (canceled)
